(19) United States
(12) Patent Application Publication
(10) Pub. No.: US 2007/0179567 A1
(43) Pub. Date: Aug. 2, 2007

(54) Customer-specific follow-up frequency

(76) Inventor: Chris J. Gennaro, Newtown, PA (US)

Correspondence Address: MEDTRONIC, INC., 710 MEDTRONIC PARKWAY NE, MINNEAPOLIS, MN, 55432-9924, US

(21) Appl. No.: 11/343,095

(22) Filed: Jan. 30, 2006

(51) Int. Cl. A61N 1/00

(52) U.S. Cl. 607/59

(57) ABSTRACT

A customer-specific follow-up frequency for patient management systems. Methods and systems are disclosed that involve providing implantable medical device (IMD) data, such as data related to an IMD, sensed physiological data, to multiple customers, such as physicians, nurses, technicians, and physician's assistants. The methods and systems allow multiple customers to receive the proper IMD data in the proper form and at the proper time for optimum patient/device management.

### BACKGROUND

Some embodiments disclosed herein relate generally to patient management systems.

Implantable medical devices (IMDs) are being offered with increasing capacity for storing physiological and device performance data. Moreover, the use of home monitoring instrumentation and equipment is gaining popularity for managing patients with chronic illnesses. Physiological sensors for monitoring various patient conditions may operate in connection with an IMD and home based instrumentation for acquiring continuous or periodic physiological data for processing and/or storage by the IMD or for clinical management. Such data may be used by the IMD in automated therapy delivery or by a clinician in diagnosing or monitoring a patient condition and in his or her therapy management.

Remote patient management systems have the potential to greatly improve efficiency in monitoring IMD patients. When IMD patients visit a clinical facility to provide data stored by the device, often no clinical action is required. The development of remote patient monitoring systems that allow IMD data to be transferred from the patient's IMD to a home monitor and from the home monitor to a central database (or from the IMD directly to the central database) may allow a customer (e.g., physician, nurse, technician, physician's assistant, etc.) to reduce the required number of scheduled office visits when a patient is doing well and still maintain an up-to-date review of device performance and stored physiological data.

The frequency with which an IMD patient provides IMD data varies by device and by customer. For example, a pacemaker or ICD patient typically sees an electrophysiologist once every three to six months, as long as he or she is not experiencing adverse symptoms. Customers other than electrophysiologists follow up with pacemaker or ICD patients at different intervals. For example, a heart failure specialist may desire the IMD data on a more frequent basis.

Moreover, the different customers may be interested in different information. Coordinating a remote patient management system and IMD to provide different information at different frequencies for different customers can prove difficult.

### BRIEF DESCRIPTION OF THE FIGURES

FIG. 1 is a schematic diagram of an exemplary remote patient management system.

FIG. 2 is a schematic diagram of a portion of the remote patient management system of FIG. 1 with an exemplary IMD shown in greater detail.

FIG. 3 is a schematic diagram of the same portion of the remote patient management system as shown in FIG. 2 with a portion of the IMD shown in greater detail.

FIG. 4 is a schematic diagram of the same portion of the remote patient management system as shown in FIG. 2 with an exemplary EMD shown in greater detail.

FIG. 5 is a schematic diagram of a remote patient management system having distributed functionality.

FIG. 6 is a flow chart showing a method of a remote patient management system receiving programming instructions.

FIG. 7 is a flow chart showing a method of a remote patient management system gathering IMD data for requesting customers.

### DETAILED DESCRIPTION OF ILLUSTRATIVE EMBODIMENTS

The following detailed description of illustrative embodiments should be read with reference to the drawings, in which like elements in different drawings are numbered identically. The drawings depict illustrative embodiments and are not intended to limit the scope of the invention. Rather, the present invention is defined solely by the claims.

FIG. 1 shows an exemplary remote patient management system 100. The remote patient management system 100 forms part of a system used for remotely managing patients prescribed with medical monitoring or therapy delivery devices. The remote patient management system 100 allows customers to monitor and treat multiple patients located at multiple locations. The exemplary remote patient management system 100 of FIG. 1 shows three locations—Location A, Location B, and Location C. Of course, the invention is not limited to three locations. Locations A, B, and C represent, e.g., the residences of three patients. The remote patient management system 100 allows patients to provide IMD data without having to visit a clinical facility. The remote patient management system 200 saves valuable time—both for the participating customers and for the patients.

Many different kinds of customers interact with the exemplary remote patient management system 100. Such customers include, for example, physicians such as hematologists, interventional cardiologists, neurologists, endocrinologists, heart failure specialists, and others. FIG. 1 shows two exemplary customers—Physician X and Physician Y. Of course the invention is not limited to two customers. Multiple customers can monitor and treat the same patient. For example, both Physician X and Physician Y can monitor the patients located at Location A, Location B, and/or Location C.

The exemplary remote patient management system 100 includes a central server 105 located at a central location. The central server 105 includes a central communication module 107, which allows the central server 105 to communicate with other components. The central communication module 107 enables the central server 105 to receive data transmissions from multiple remote medical devices for enabling remote patient management of a population of patients. The central communication module 107 enables the central server 105 to provide information, such as instructions, to medical devices at multiple locations. The central server 105 interacts with various types of equipment at each location through a network 110. The network 110 can be a local area network (LAN), a wide area network (WAN), or other suitable telecommunications network, including the Internet. The central server 105 includes a processor that operates with an associated central database. The central database may store patient data and/or programs and algorithms used by the processor in performing patient management operations (e.g., programming and/or interrogating IMDs). The central database can include electronic medical records in a relational database and can include data files and code used for controlling communication with external components.

An IMD 115, 120, 125 (implanted inside of a patient) and an EMD 130, 135, 140 are located remotely at each of Locations A, B, and C of the exemplary remote patient management system 100. The IMD 115, 120, 125 can be a cardiac stimulation device (e.g., a pacemaker), cardioverter/defibrillator (ICDs), a cardiac monitor, a hemodynamic monitor, a neuromuscular stimulator, a drug delivery device, or other IMD. The IMD 115, 120, 125 at one location can be the same as or different than the IMD 115, 120, 125 at the other locations. The EMD 130, 135, 140 can be a remote home monitor, programmer, or other EMD. The EMD 130, 135, 140 collects various physiological indicators from IMD patients, such as weight, systemic blood pressure, symptoms, and others. The EMD 130, 135, 140 at one location can be the same or different than the EMD 130, 135, 140 at the other locations.

The central server 105, the network 110, the IMD 115, 120, 125, and the EMD 130, 135, 140 can be configured to communicate in a variety of ways. In one example, the IMD 115, 120, 125 is configured to communicate with the EMD 130, 135, 140, which is configured to communicate with the central server 105 via the network 110. In some embodiments, the IMD 115, 120, 125 is configured to communicate with the central server 105 directly through the network 110. In some embodiments, the central server 105 communicates directly with either the IMD 115, 120, 125 or the EMD 130, 135, 140 without use of the network 110. Communication between the central server 105, the network 110, the IMD 115, 120, 125, and/or the EMD 130, 135, 140 can be, for example, bi-directional. The communication configuration can be the same or different with respect to Locations A, B, and C. In other words, in some embodiments, the central server 105 is configured to communicate with Location B's IMD 120 through the network 110 while being configured to communicate with Location C's IMD 125 through the EMD 140 without the use of the network 110.

The content of the information being communicated between the central server 105, the EMD 130, 135, 140, and the IMD 115, 120, 125 varies according to the particular application. In some applications, the IMD 115, 120, 125 provides IMD data to the central server 105. For example, the IMD 115, 120, 125 can gather and/or store information related to parameters such as device performance and various physiological indicators of a patient (e.g., heart rhythm, blood pressure, respiration, patient activity level, heart wall motion, blood chemistry, and the like). The IMD 115, 120, 125 gathers and/or stores such IMD data on a continuous or periodic basis. In some embodiments, the IMD 115, 120, 125 communicates some or all of that IMD data to either the EMD 130, 135, 140 or the central server 105. In some embodiments, the EMD 130, 135, 140 and/or the central server 105 pull such IMD data from the IMD 115, 120, 125. In some embodiments, the IMD 115, 120, 125 pushes such IMD data to the EMD 130, 135, 140 and/or the central server 105. The EMD 130, 135, 140, for example, communicates some or all of the IMD data received from the IMD 115, 120, 125 to the central server 105. In some embodiments, the EMD 130, 135, 140 performs one or more processing operations on the IMD data received from the IMD 115, 120, 125. In some embodiments, the central server 105 pulls such IMD data from the EMD 130, 135, 140. In some embodiments, the EMD 130, 135, 140 pushes such IMD data to the central server 105.

In some applications, the central server 105 provides information to the IMD 115, 120, 125 and/or to the EMD 130, 135, 140. In one example, Physician X enters instructions related to, e.g., therapy, operating parameters, transfer code, or other instructions, into the central server 105. The central server 105 communicates those instructions to the IMD 115, 120, 125 and/or the EMD 130, 135, 140, either through the network 110 or directly. In some embodiments, the EMD 130, 135, 140 and/or the IMD 115, 120, 125 pull such information from the central server 105. In some embodiments, the central server 105 pushes such information to the EMD 130, 135, 140 and/or the IMD 115, 120, 125.

The IMD 115, 120, 125 can be configured to communicate with the EMD 130, 135, 140 through telemetry antennas. In one example, the IMD 115 at Location A includes an IMD antenna, and the corresponding EMD 130 includes an EMD antenna. In some embodiments, the EMD antenna is contained within the EMD 130. In such embodiments, the IMD 115 communicates with the EMD 130 when the EMD 130 is positioned close to the patient's skin overlying the IMD 115. In such embodiments, the IMD patient manually positions the EMD 130 over the IMD 115. In some embodiments, the EMD antenna is located on the case of the EMD 130. In such embodiments, the IMD 115 may communicate with the EMD 130 even when the EMD 130 is located some distance (e.g., several meters) away from the IMD 115. Such embodiments implement long-range telemetry systems. Some long-range telemetry systems allow the IMD 115 to communicate with the EMD 130 without any patient intervention (e.g., passive telemetry transmission). In other words, as long as the IMD patient is within a communication range of the EMD 130, he or she may be engaged in ordinary activities during a telemetry transmission and may be completely unaware that such transmission is taking place. The IMDs 120, 125 and the EMDs 135, 140 at Locations B and C, respectively, can incorporate the same or different communication configurations. In other words, in certain embodiments, the IMD 120 and the EMD 135 at Location B incorporate a manual, short-range telemetry system, while the IMD 125 and the EMD 140 at Location C incorporate a long-range, passive telemetry system.

In some embodiments, the IMD 115, 120, 125 and the EMD 130, 135, 140 communicate bi-directionally and in a variety of wireless formats. For example, communication may be via RF. In a transmission from the IMD 115, 120, 125 to the EMD 130, 135, 140 (an uplink telemetry transmission), the IMD antenna operates as a telemetry transmitter antenna, and the EMD antenna operates as a telemetry receiver antenna. Conversely, in a transmission from the EMD 130, 135, 140 to the IMD 115, 120, 125 (an downlink telemetry transmission), the EMD antenna operates as a telemetry transmitter antenna, and the IMD antenna operates as a telemetry receiver antenna. Both the IMD antenna and the EMD antenna can be coupled to a transceiver including a transmitter and a receiver.

The content of the information being communicated between the EMD 130, 135, 140 and the IMD 115, 120, 125 varies according to the particular application. In some embodiments, the EMD 130, 135, 140 simply relays communications between the IMD 115, 120, 125 and the central server 105. In some embodiments, the EMD 130, 135, 140 and the IMD 115, 120, 125 communicate between each other without regard for the central server 105. In such embodiments, the EMD 130, 135, 140 provides instructions concerning, e.g., therapy, operating parameters, transfer code, or other instructions, to the IMD 115, 120, 125 in response to IMD data provided by the IMD 115, 120, 125.

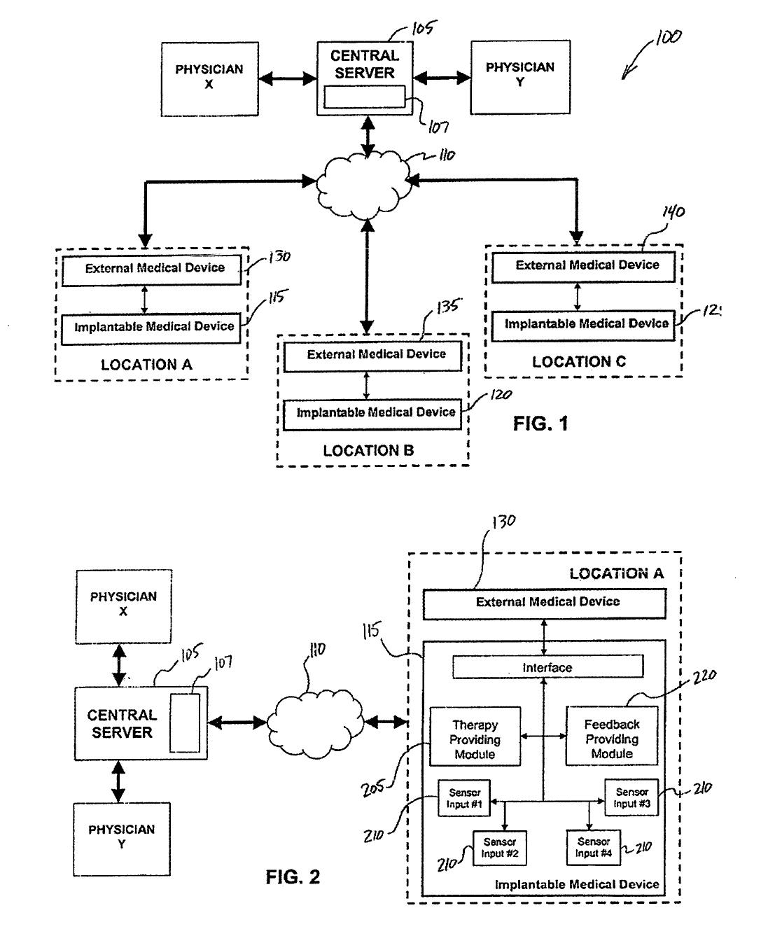

FIG. 2 shows a portion of the exemplary remote patient management system 100 of FIG. 1 with the IMD 115 of Location A shown in greater detail. The IMD 115 includes a therapy providing module 205. The therapy providing module 205 can provide various kinds of therapy, such as a cardiac stimulation (e.g., various types of pacing), defibrillation, cardiac monitoring, hemodynamic monitoring, neuromuscular stimulation, drug delivery, or other suitable therapy. Therapy providing modules and corresponding therapy providing devices are well known in the art.

The IMD 115 includes one or more sensor inputs 210. The sensor inputs 210 correspond to various kinds of sensed physiological data from, e.g., the patient. Such sensed physiological data includes, for example, data related heart rhythm, blood pressure, respiration, activity level, heart wall motion, and/or blood chemistry. The sensor inputs 210 can come from a many kinds of sensors, such as pressure sensors, optical sensors, biochemical sensors, protein sensors, motion sensors (e.g., accelerometers or gyroscopes), temperature sensors, chemical sensors (e.g., pH sensors), genetic sensors, and the like. The data provided through the sensor inputs 210 can be stored in a sensed physiological data repository. The IMD 115 shown in FIG. 2 includes four sensor inputs 210. It is contemplated that other numbers of sensor inputs could be provided.

The IMD 115 includes a feedback providing module 220. The feedback providing module 220 can be configured to provide IMD data to the central server 105 at appointed times. For example, Physician X may indicate that he or she would like IMD data corresponding to a set of desired parameters (e.g., blood pressure and heart rhythm) a particular time. Physician X's desires can be translated into follow-up instructions that define a set of data fields (e.g., parameters, patient-identifying information, device-identifying information, etc.) and include a set of timing information corresponding to the time at which Physician X would like the IMD data. The follow-up instructions can be provided to the central server 105, EMD 130, or the IMD 115. In embodiments in which the follow-up instructions are provided to the central server 105, the central server 105 then provides those follow-up instructions to the IMD 115 (directly, through the network 110, through the EMD 130, or through a combination thereof). The feedback providing module 220 of the IMD can be configured to provide the IMD data that falls within the set of data fields at the desired time in response to those follow-up instructions. In the event that Physician Y desired a set of IMD data at a particular time (whether that set of IMD data and/or that time are the same as or different than Physician X's desires), the feedback providing module 220 can be equipped to provide IMD data responsive to Physician Y's desires as well. The feedback providing module 220 can be equipped to provide responsive IMD data to several different customers.

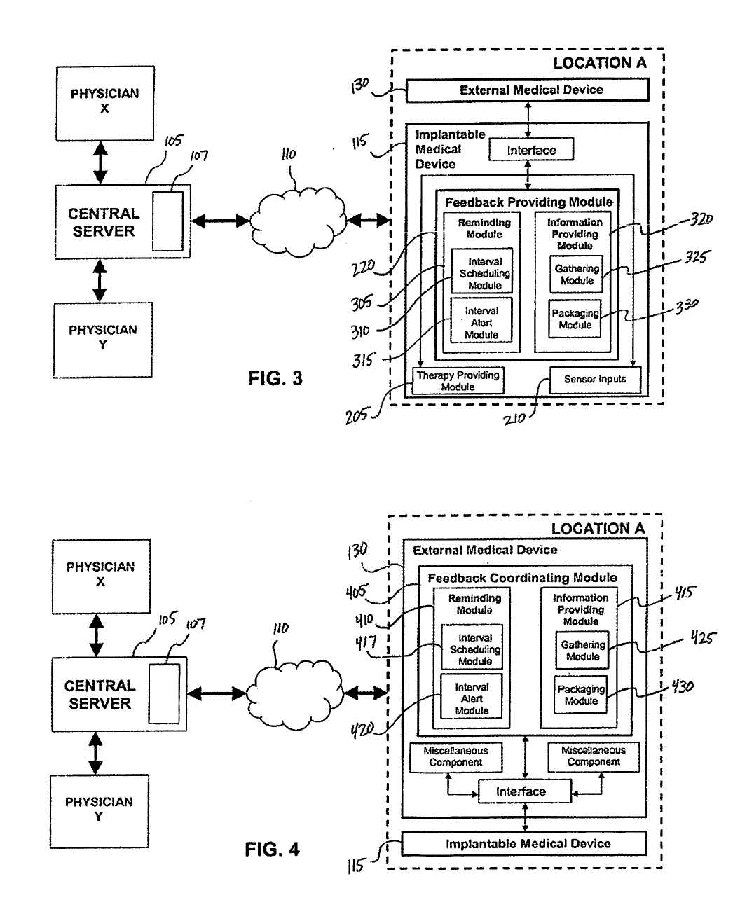

FIG. 3 shows the same portion of the remote patient management system 100 of FIG. 1 as shown in FIG. 2, with the feedback providing module 220 of Location A's IMD 115 shown in greater detail. Following on the example of the previous paragraph, the feedback providing module 220 receives follow-up instructions from, e.g., Physician X. Follow-up instructions include, for example, timing information, such as information related to the date, or a series of dates, on which Physician X desires the IMD data corresponding to the desired parameters. For example, an electrophysiologist may desire IMD data related to a pacemaker or an ICD approximately every three to six months. Timing information coming from an electrophysiologist's follow-up instructions can include a series of follow-up dates spaced apart by approximately three to six months. A heart failure specialist, on the other hand, may desire IMD data on a more frequent basis. Timing information coming from a heart failure specialist's follow-up instructions may include a series of follow-up dates spaced apart by approximately 1-4 weeks. The feedback providing module 220 includes a reminding module 305, which triggers the IMD 115 to provide the appropriate IMD data at times corresponding to the timing information. The reminding module 305 includes an interval scheduling module 310 for scheduling follow-up reminders. Upon receiving the timing information, the interval scheduling module 310 can schedule a follow-up reminder by creating an interval profile that includes Physician X's name and the relevant timing information. That interval profile can be stored in an interval repository. The reminding module 305 includes an interval alert module 315. The interval alert module 315 interacts with a timer associated with the interval repository. The timer can trigger interval alert module 315 to notify the IMD 115 that Physician X is due to receive IMD data.

In certain embodiments, the follow-up instructions referenced in the previous two paragraphs include a set of data fields (e.g., parameters, patient-identifying information, device-identifying information, etc.) corresponding to the IMD data. In one example, an electrophysiologist's set of data fields includes, for example, device-identifying data and device-performance data. The feedback providing module 220 includes an information providing module 320, which is responsible for providing the IMD data that falls within the set of data fields, in the desired form, to Physician X. The information providing module 320 includes a gathering module 325. When the feedback providing module 220 receives follow-up instructions, the gathering module 325 may create an information profile that includes Physician X's name, the set of data fields, and the desired form of the IMD data falling within those data fields. In some embodiments, the desired form of the IMD data is the current IMD data falling within the data fields along with the IMD data that fell within the data fields on Physician X's most recent follow-up date. For example, an electrophysiologist interested in certain parameters of device performance may desire the most current device performance data and the device performance data that he or she received three months earlier for purposes of comparison. That information profile can be stored in an IMD data repository.

When the IMD 115 is notified that Physician X is due to receive IMD data by the reminding module 305, the gathering module 325 is triggered in certain embodiments to retrieve the appropriate IMD data from, e.g., the sense physiological data repository. The information providing module 320 includes a packaging module 330, which is responsible for providing the desired IMD data in the desired form. In one example, the packaging module 330 retrieves the IMD data that fell within the data fields on Physician X's most recent follow-up date and packages it together with the current relevant IMD data. The gathering module 325 provides the content of the desired IMD data to the packaging module 330. After the packaging module 330 manipulates the IMD data into the desired form, the packaging module 330 triggers the IMD 115 to provide the appropriate IMD data to the central server 105 (through the EMD 130, through the network 110, directly to the central server 105, or by combinations thereof). The information providing module 320 performs similarly in some embodiments to provide IMD data to other customers, such as Physician Y.

The IMD 115 shown in FIG. 3 includes several components. IMDs may include a greater or lesser number of components than those shown. In IMD embodiments, the functions performed by multiple components in the IMD 115 discussed herein are combined into fewer components, such as one component. In one example, the multiple functions performed by the reminding module 305 and the information providing module 320 discussed herein are performed by a single component designed to collect and package the relevant IMD data and to do so on the desired date(s). In some IMD embodiments, the functions performed by one component in the IMD 115 discussed herein are performed by multiple components. The multiple functions of the gathering module 325 discussed herein are, for example, performed by a component designed to create information profiles, or a component designed to gather IMD data or other miscellaneous components. Some IMD embodiments include additional components to provide additional functionality. In one example, a component is provided that is dedicated to providing IMD data directly to the therapy providing module 205 to impact the therapy being provided to the IMD patient. Components of IMDs can perform the functions discussed herein in various ways. The ways by which the components of the IMD 115 discussed herein are provided only for illustration.

FIG. 4 shows the same portion of the remote patient management system 100 as shown in FIG. 2, with Location A's EMD 130 shown in greater detail. The EMD 130 includes a feedback coordinating module 405, which allows the EMD 130 to provide the appropriate IMD data in the appropriate form on the appointed date(s). The feedback coordinating module 405 includes a reminding module 410 and an information providing module 415. The reminding module 410 includes an interval scheduling module 417 and an interval alert module 420. The information providing module 415 includes a gathering module 425 and a packaging module 430. The reminding module 410 and the information providing module 415, and their respective constituent modules, can have substantially the same functionality as corresponding components discussed in connection with FIG. 3.

The EMD 130 shown in FIG. 4 includes several components. It is contemplated though that EMDs could include a different number of components than those shown. In EMD embodiments, the functions performed by multiple components in the EMD 130 discussed herein are combined into fewer components, such as one component. Examples include the examples provided above in connection with FIG. 3. In some EMD embodiments, the functions performed by one component in the EMD 130 discussed herein are performed by multiple components. Examples include the examples provided above in connection with FIG. 3. Some EMD embodiments include additional components to provide additional functionality, such as those examples provided in connection with FIG. 3. Components of EMDs perform the functions discussed herein in various ways. The ways by which the components of the EMD 130 discussed herein are provided only for illustration.

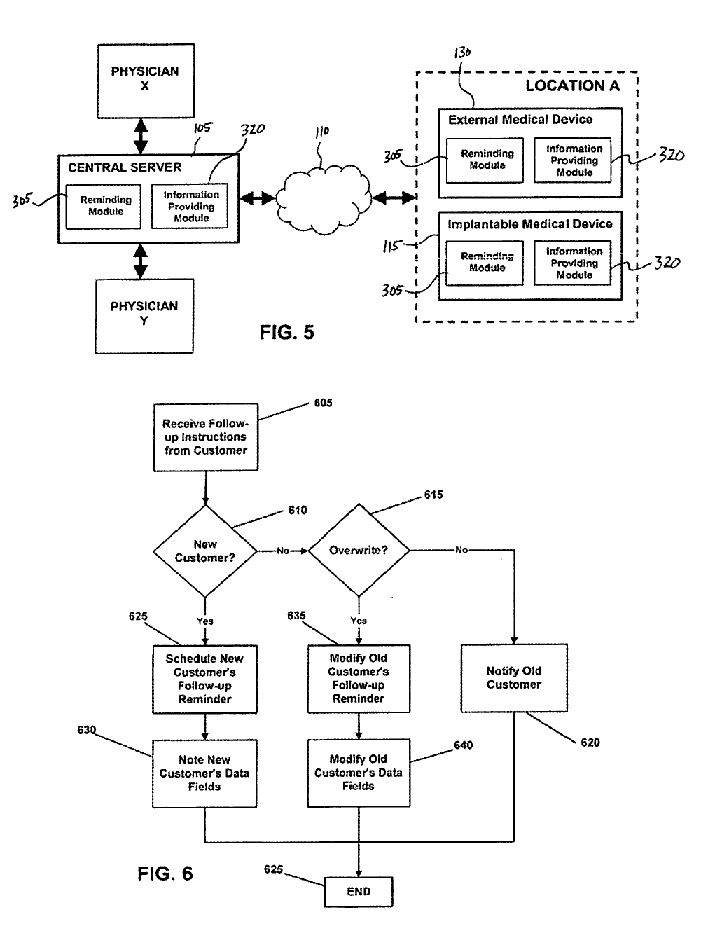

In the exemplary remote patient management system 100, the functionality of providing the desired IMD data in the desired form on the desired date(s) can reside in the central server 105, in the EMD 130, in the IMD 115, or it can be spread across all three components. In one example, the IMD 115 takes care of setting follow-up reminders based on the timing information and provides more IMD data than falls within the relevant set of data fields to the EMD 130. The EMD 130 then provides only the IMD data that falls within the relevant data fields to the central server 105. The central server 105 can then package the IMD data into the appropriate form to provide to the requesting customer. This way, the remote patient management system 100 can include an EMD 130 with a feedback coordinating module 405, an IMD 115 with the feedback providing module 220, or some combination of both. In another example, the central server 105 takes care of setting follow up reminders based on the timing information, pulls the relevant IMD data from the IMD 115, and manipulates the IMD data into the desired form to provide to the corresponding customer. In another example, shown in FIG. 5, an information providing module 320 and/or a reminding module 305 is located in the central server 105, and an information providing module 320 and/or a reminding module 305 is located at Location A in the EMD 130 and/or the IMD 115. Several other configurations are possible.

FIG. 6 shows an exemplary method that can be implemented in connection with programming a remote patient management system to provide IMD data to a requesting customer. The system receives follow-up instructions from a customer (605). As discussed elsewhere herein, embodiments of the invention permit multiple customers to use the same remote patient management system. The system inquires whether the customer providing the follow-up instructions is a new customer (610). In the event that the customer is a new customer, the system schedules a follow-up reminder associated with that customer (625). The follow-up reminder can be similar to other follow-up reminders discussed elsewhere herein. For a new customer, the system notes such customer's data fields (630). The system notes, for example, such customer's data fields, along with customer-identifying information, in storage. The data fields can be similar to data fields discussed elsewhere herein. In some embodiments, the system schedules a follow-up reminder (625) before noting data fields (630). In some embodiments, the system notes data fields (630) before scheduling a follow-up reminder (625). After scheduling a follow-up reminder (625) and noting data fields (630), the system is ready to respond to the received follow-up instructions at an appropriate time, and the method shown in FIG. 6 comes to an end (625).

In the event that the system determines that the customer is not a new customer, the system determines whether to overwrite existing information related to the old customer (615). The system makes such determination based on a variety of factors, such as receipt of further input from the customer. In the event that the system of does not overwrite the existing information related to the old customer, the system notifies the old customer that the new follow-up instructions are not properly received (620), and the method shown in FIG. 6 comes to an end (625). In the event that the system does overwrite the existing information related to the old customer, the system modifies the old customer's follow-up reminder (635). The system modifies the old customer's follow-up reminder (635) by any of the ways discussed elsewhere herein or by any other suitable way. In the event that the system does overwrite the existing information related to the old customer, the system modifies the old customer's data fields (640). In some embodiments, the system modifies the old customer's follow-up reminder (635) before modifying the old customer's data fields (640). In some embodiments, the system modifies the old customer's data fields (640) before modifying the old customer's follow-up reminder (635). After modifying the old customer's follow-up reminder (635) and modifying the old customer's data fields (630), the system is ready to respond to the received follow-up instructions at an appropriate time, and the method shown in FIG. 6 comes to an end (625).

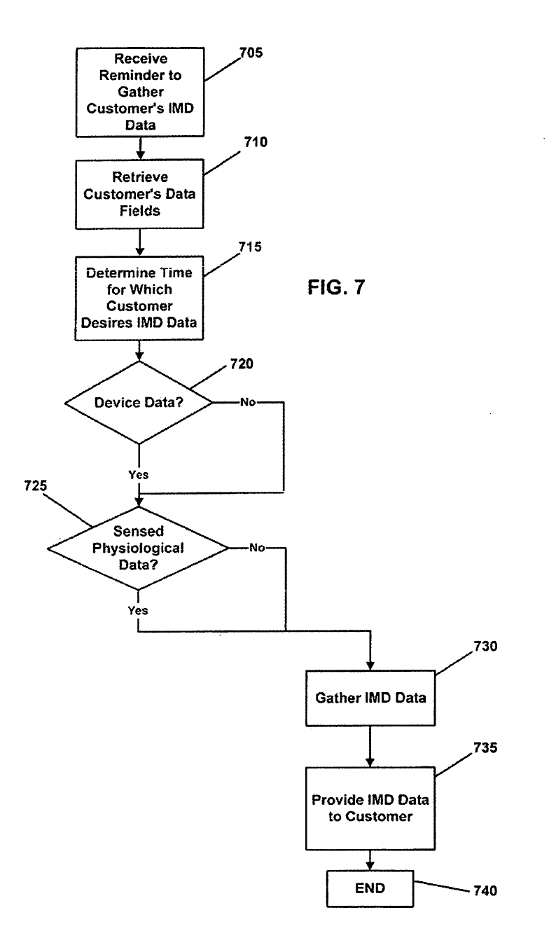

FIG. 7 shows an exemplary method implemented in connection with a remote patient management system for responding to the received follow-up instructions at an appropriate time. A follow-up reminder set for a particular customer (see steps 625 and 635 of FIG. 6) is received by the system to trigger the system to begin gathering that customer's IMD data (705). The follow-up reminder triggers the system to begin gathering in any of the ways discussed herein or in any other suitable way. After being notified that a customer is due to receive IMD data, the system retrieves that customer's data fields (710). In some embodiments, the customer desires IMD data only for particular time periods. For example, a customer may desire current relevant IMD data along with relevant IMD data from the customer's most recent follow-up date. In such embodiments, the system determines the time for which the customer desires IMD data (715). In some embodiments, the data fields include data related to the device itself and/or to physiological data sensed by the device and/or other sensors. The system inquires whether the data fields include device data (720). The system inquires whether the data fields include sensed physiological data (725). In some embodiments, the device data inquiry (720) occurs before the sensed physiological data inquiry (725). In some embodiments, the sensed physiological data inquiry (725) occurs before the device data inquiry (720).

After determining the type of IMD data desired by the requesting customer, the system is, for example, configured to provide such IMD data to the customer. The system can gather IMD data (730) from a variety of sources such as those discussed elsewhere herein. The system can do so by any of the methods discussed herein, such as by comparing all of the IMD data to the data fields and selecting only the IMD data that falls within the data fields. The system then provides the IMD data to the customer (735) in any of the ways discussed herein or in any other suitable way, and the method shown in FIG. 7 comes to an end (740). The order of steps provided in the methods shown in FIGS. 6-7 is provided for purposes of illustration only, and other orders that achieve the functionality recited are within the scope of the present invention. Any of the functionality discussed anywhere in this disclosure may be implemented in either of the methods shown in FIGS. 6-7.

Embodiments of the present invention may have one or more of the following advantages. In some embodiments, multiple customers are able to receive the proper IMD data in the proper form and at the proper time for optimum patient/device management. In some embodiments, multiple customers are able to receive desired IMD data at different intervals. In some embodiments, the patient need not visit a clinical facility in order to provide his or her IMD data to multiple customers. In some embodiments, the patient can manually upload his or her IMD data to multiple customers. In some embodiments, the patient can upload his or her IMD data to multiple customers simply by being within a communication range of an external medical device (EMD). In such embodiments, the IMD may push IMD data to either in EMD or a central server. In such embodiments, an EMD or a central server may pull IMD data from the IMD.

Thus, embodiments of the present invention are disclosed. One skilled in the art will appreciate that the present invention can be practiced with embodiments other than those disclosed. The disclosed embodiments are presented for purposes of illustration and not limitation, and the present invention is limited only by the claims that follow.

What is claimed is:

1. A remote patient management system of patients with an implantable medical device (IMD), the system comprising:
a central communication module located in a central server at a central location;
an information providing module configured to gather IMD data associated with a patient and falling within a first set of data fields based on a first customer's follow-up instructions, the first customer's follow-up instructions defining the first set of data fields and including a first set of follow-up timing information, the information providing module configured to gather IMD data associated with the patient and falling within a second set of data fields based on a second customer's follow-up instructions, the second customer's follow-up instructions defining the second set of data fields and including a second set of follow-up timing information, and the information providing module configured to provide the gathered IMD data to the central communication module; and
a reminding module configured to trigger the information providing module to gather the IMD data within the first set of data fields based on the first set of follow-up timing information, and the reminding module configured to trigger the information providing module to gather the IMD data within the second set of data fields based on the second set of follow-up timing information.

2. The remote patient management system of claim 1, wherein the information providing module and the reminding module are located in an implantable medical device at a remote location.

3. The remote patient management system of claim 1, wherein the information providing module and the reminding module are located in an external medical device at a remote location.

4. The remote patient management system of claim 1, wherein one of the information providing module and the reminding module is located in the central server and the other of the information providing module and the reminding module is located at a remote location.

5. The remote patient management system of claim 1, wherein the first set of timing information includes multiple follow-up dates, the IMD data being gathered for the first customer on the multiple follow-up dates.

6. The remote patient management system of claim 5, wherein the IMD data gathered for the first customer on a second one of the follow-up dates includes IMD data generated after a first one of the follow-up dates.

7. The remote patient management system of claim 1, wherein the data fields are physiological parameters of the patient.

8. The remote patient management system of claim 7, wherein the physiological parameters include cardiac rhythm parameters of the patient.

9. A computer-readable medium programmed with instructions for performing a method of providing IMD data to multiple customers, the medium comprising instructions for causing a programmable processor to:
receive follow-up instructions from a first customer and a second customer, the first customer's follow-up instructions including a first set of follow-up dates and the second customer's follow-up instructions including a second set of follow-up dates;
gather IMD data for the first customer on one of the follow-up dates in the first set, the first customer's IMD data including data generated after the follow-up date immediately preceding the one of the follow-up dates in the first set, the first customer's IMD data being associated with a patient;
provide the first customer's IMD data to the first customer;
gather IMD data for the second customer on one of the follow-up dates in the second set, the second customer's IMD data including data generated after the follow-up date immediately preceding the one of the follow-up dates in the second set, the second customer's IMD data being associated with the patient; and
provide the second customer's IMD data to the second customer.

10. The medium of claim 9, wherein the medium and the processor are located in one of an implantable medical device, an external medical device, and a central server.

11. The medium of claim 9, further comprising instructions to
receive follow-up instructions from a third customer, the third customer's follow-up instructions including a third set of follow-up dates;
gather IMD data for the third customer on one of the third set of follow-up dates, the third customer's IMD data including data generated after the follow-up date immediately preceding the one of the third set of follow-up dates, the third customer's IMD data being associated with the patient; and
provide the third customer's IMD data to the third customer.

12. The medium of claim 9, wherein the first customer's IMD data excludes IMD data generated before the follow-up date immediately preceding the one of the first set of follow-up dates.

13. The medium of claim 9, wherein the IMD data is gathered from an IMD implanted in the patient.

14. The medium of claim 9, wherein the IMD data is gathered from an external medical device adapted for wireless communication with an IMD implanted in the patient.

15. The medium of claim 9, wherein the follow-up instructions from the first customer includes a first group of data fields of IMD data, and wherein the IMD data gathered for the first customer is within the first group of data fields.

16. The medium of claim 15, wherein the follow-up instructions from the second customer includes a second group of data fields of IMD data, and wherein the IMD data gathered for the second customer is within the second group of data fields.

17. A computer-implemented method for providing IMD data to multiple customers, the method comprising:
receiving follow-up instructions from a first customer and a second customer, the first customer's follow-up instructions including a first set of follow-up timing information and defining a first set of data fields of IMD data to gather, the second customer's follow-up instructions including a second set of follow-up timing information and defining a second set of data fields of IMD data to gather;
scheduling a first follow-up reminder based on the first set of follow-up timing information and a second follow-up reminder based on the second set of follow-up timing information;
responding to the first follow-up reminder by gathering IMD data falling within the first set of data fields for the first customer;
providing the first customer's IMD data to the first customer;
responding to the second follow-up reminder by gathering IMD data falling within the second set of data fields for the second customer; and
providing the second customer's IMD data to the second customer.

18. The computer-implemented method of claim 17, wherein scheduling the first follow-up reminder occurs in one of an implantable medical device and an external medical device, and gathering the first set of IMD data occurs in the other of the implantable medical device and the external medical device.

19. The computer-implemented method of claim 17, wherein the IMD data falling within the first set of data fields includes sensed physiologic data regarding the patient.

20. The computer-implemented method of claim 17, further comprising scheduling a series of follow-up reminders based on the first set of timing information, wherein the first set of timing information includes multiple follow-up dates.
